(19) United States
(12) Patent Application Publication
(10) Pub. No.: US 2014/0148860 A1
(43) Pub. Date: May 29, 2014

(54) Expanding Blade Screw Retention System

(71) Applicant: Jim A. Rinner, Franksville, WI (US)

(72) Inventor: Jim A. Rinner, Franksville, WI (US)

(21) Appl. No.: 14/091,604

(22) Filed: Nov. 27, 2013

Related U.S. Application Data
(60) Provisional application No. 61/730,118, filed on Nov. 27, 2012.

(51) Int. Cl. A61B 17/86; A61B 17/80

(52) U.S. Cl. CPC A61B 17/8685; A61B 17/8057
USPC 606/289; 606/302

(57) ABSTRACT

Disclosed are devices, systems, methods and surgical techniques for the retention of bone screws used in surgical applications. Various aspects of the invention include features to retain bone screws that are placed in an elongated plate and into a bony member to create a rigid construct and stabilize the member for the purpose of fusion. When the screws are placed through the plate, the retention systems can be engaged to prevent the screws from backing out.

### CROSS-REFERENCE TO OTHER APPLICATIONS

This application claims the benefit of U.S. Provisional Patent Application Ser. No. 61/730,118 entitled “Expanding Blade Screw Retention System,” filed Nov. 27, 2012, the disclosure of which is incorporated herein by reference in its entirety.

### TECHNICAL FIELD

The present invention relates to devices, instrumentation, systems and methods that facilitate the retention of bone screws and/or other components in constructs used for various surgical applications. More particularly, the invention includes features that selectively retain bone screws or other anchoring devices that have been introduced through a base member and/or elongated plate and into a patient's bony anatomy. When the screws are advanced through the base member, the retention systems can be engaged, thereby preventing the screws from “backing out” of the member and/or bony anatomy. In various embodiments, the base member can create a rigid construct and stabilize the patient's anatomy for the purpose of fusion.

### BACKGROUND OF THE INVENTION

The invention relates generally to the field of orthopedic surgery and can be used in various applications that use bone screws. The bone screws are placed through many devices and then into the patient's bone. Because the bony elements are constantly moving there are forces applied to the fusion construct that can cause the screws to loosen. If the screws were to loosen to a degree that they would back out of the construct then they could become dislodged and cause harm to the fusion sight or to the patient. For this reason many have endeavored to use various systems to lock or retain the screw with various amounts of success. Many systems use additional components that need to be attached after the screws are placed which could cause misplacement of the retention device and failure to retain the screw.

### SUMMARY OF THE INVENTION

The invention uses an anterior cervical plate as the base substrate for the retention system but the invention can be used in other orthopedic applications as well. As mentioned earlier many systems use an additional construct that is added after the placement of the bone screws. The present invention does not rely on the correct placement of a retention system after the bone screws are placed but the invention's retention components are placed into position by the manufacturer of the device and are completely assembled prior to the surgery thus reducing the chances of misplacement and reducing the amount of time to perform the surgery.

In one embodiment, the invention has three components; the base, the dual retention blade and retention screw. As stated earlier the base can be any number of orthopedic fusion devices such as a vertebral body replacement, bone plate or as in the current description an anterior cervical plate, where the plate can be modified to include a recessed surface where the surface will accommodate the size and configuration of the retention blade, and/or may have at least one bone screw threaded hole. In addition, the retention blade may be designed as an elongated plate with a notch and a female threaded hole that receives the retention screw. The notch may allow the dual retention blade to be split into at least two flexible arms.

In another embodiment, the invention has three components; the base, the single retention blade and retention screw. The base may be specifically designed to include a recessed surface where the surface will accommodate the size and configuration of the retention blade. In addition, the single retention blade may be designed as an elongated plate with a notch and a female threaded hole that receives the retention screw. The notch may allow the single retention blade to be split into at least one flexible arms.

In another embodiment, the retention screw may be designed with different surfaces and configurations. For example, the retention screw may include a plurality of increasing diameter surfaces, where the surgeon may rotate the retention screw until the increased diameters expand the flexible arms of a dual and/or single retention blade into the base bone screw threaded hole as desired. In another example, the retention screw may have different configurations of surfaces, where the surgeon may rotate the retention screw until it contacts the different configurations of surfaces (i.e., a camming or other action) to expand the flexible arms of a dual and/or single retention blade into the base bone screw threaded hole as desired. Furthermore, when the retention screw is advanced to the final position on the different surfaces or configurations, the retention blade and/or the retention screw can have an audible, tactile and/or visual features to allow the components to snap, or spring into causing an audible click and a tactile feedback on the driver. The surgeon will also be able to clearly see the retention blade covering the screw or screws.

In another embodiment, the base, bone screws, retention blade and/or the retention screw may be pre-assembled by the manufacturer prior to the insertion of cervical spinal fixation system to the targeted bone segment. This allows the surgeon to attach the cervical spinal fixation system to in-situ to the targeted bone segment and secure the bone screws in a single operation.

### BRIEF DESCRIPTION OF THE DRAWINGS

The present invention will be more fully understood with reference to the detailed description and the accompanying drawings.

FIG. 1 is an isometric view of one embodiment of a cervical spinal fixation system;

FIG. 2 is an isometric exploded view of the cervical spinal fixation system in FIG. 1;

FIG. 3 is a top view of one embodiment of an anterior cervical plate;

FIG. 4 is a side view of the anterior cervical plate of FIG. 3;

FIG. 5 is a sectioned view along lines 5-5 of the anterior cervical plate in FIG. 3;

FIG. 6 is a bottom view of the anterior cervical plate of FIG. 3;

FIG. 7 is an enlarged view of area 7 of the anterior cervical plate in FIG. 3;

FIG. 8 is an enlarged partial sectioned along lines 8-8 of the anterior cervical plate in FIG. 4;

FIG. 9 is a top view of one embodiment of a dual-bladed retention blade;

FIG. 10 is an end view of the dual-bladed retention blade of FIG. 9;

FIG. 11 is a top view of one embodiment of a retention screw;

FIG. 12 is a side view of the retention screw of FIG. 11;

FIG. 13 is an enlarged view of area 13 the retention screw in FIG. 12;

FIG. 14 is a top view of one embodiment of a bone screw;

FIG. 15 is a side view of the bone screw of FIG. 14;

FIG. 16 is a top view of one embodiment of the retention subassembly demonstrating the neutral position of the retention blade;

FIG. 17 is a side view of the retention subassembly of FIG. 16 demonstrating the neutral position of the retention blade;

FIG. 18 is an enlarged view of area 18 of the retention subassembly in FIG. 17;

FIG. 19 is a top view of the retention subassembly demonstrating the retention position of the retention blade;

FIG. 20 is a side view of the retention subassembly demonstrating the retention position of the retention blade;

FIG. 21 is an enlarged view of area 21 of the retention subassembly in FIG. 20;

FIG. 22 is a top view of one embodiment of a cervical spinal fixation system highlighting the retention features in the neutral position;

FIG. 23 is a partial sectioned end view along lines 23-23 of the cervical spinal fixation system in FIG. 22;

FIG. 24 is an enlarged view of area 24 of the retention subassembly in FIG. 23;

FIG. 25 is an enlarged view of area 25 of the retention subassembly in FIG. 23;

FIG. 26 is a top view of one embodiment of a cervical spinal fixation system highlighting the retention features in the retention position;

FIG. 27 is a partial sectioned end view of the cervical spinal fixation system along lines 26-26 in FIG. 25;

FIG. 28 is an enlarged view of area 28 of the retention subassembly in FIG. 27;

FIG. 29 is a top view of an alternate embodiment of a cervical spinal fixation system containing a single retention blade embodiment in the retention position;

FIG. 30 is an isometric view of the cervical spinal fixation system containing a single retention blade of FIG. 29 in the retention position;

FIG. 31 is a top view of an alternate embodiment of a cervical spinal fixation system containing a single retention blade in the neutral position;

FIG. 32 is an exploded isometric view of the cervical spinal fixation system containing a single retention blade of FIG. 31 in the neutral position; and

FIG. 33 is a top view of the single-bladed retention blade.

### DETAILED DESCRIPTION OF THE PREFERRED EMBODIMENTS

For the purpose of demonstrating how the invention functions it is shown as part of an anterior cervical plate. As stated earlier, those familiar with the art should clearly understand that various embodiments of an expanding blade screw retention system can be incorporated into many different types of devices, including those involving bone screws or other anchoring mechanisms, and therefore is not limited to the use of an anterior cervical plate only.

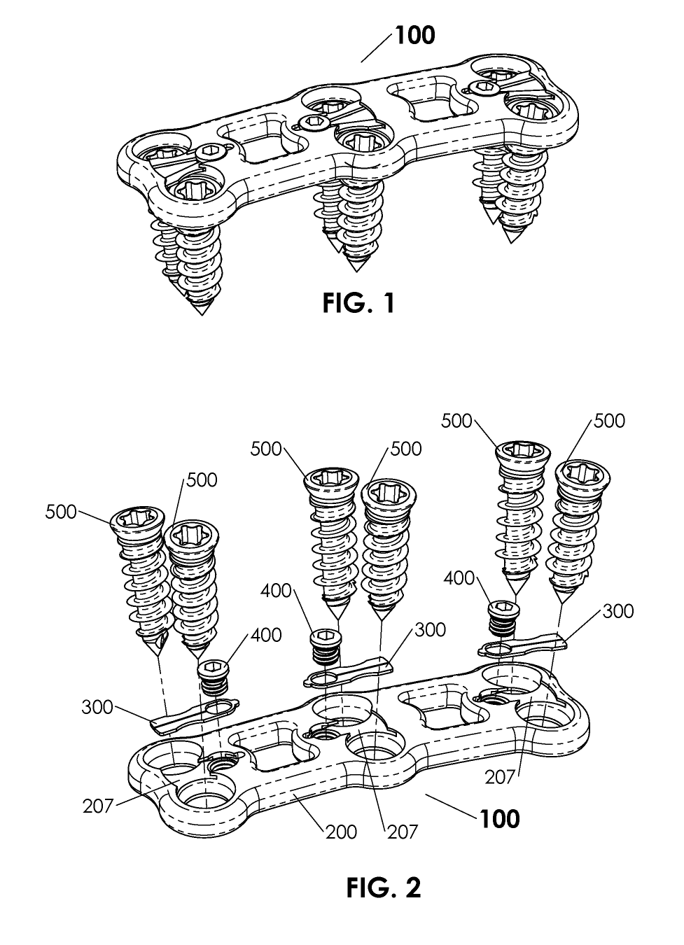

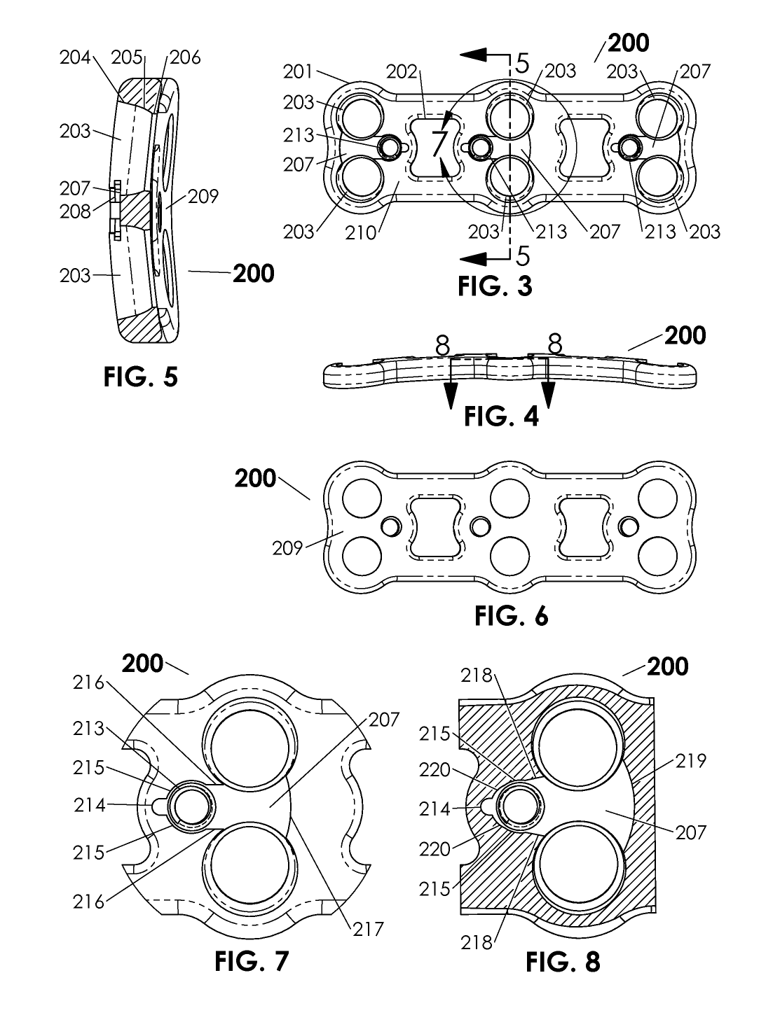

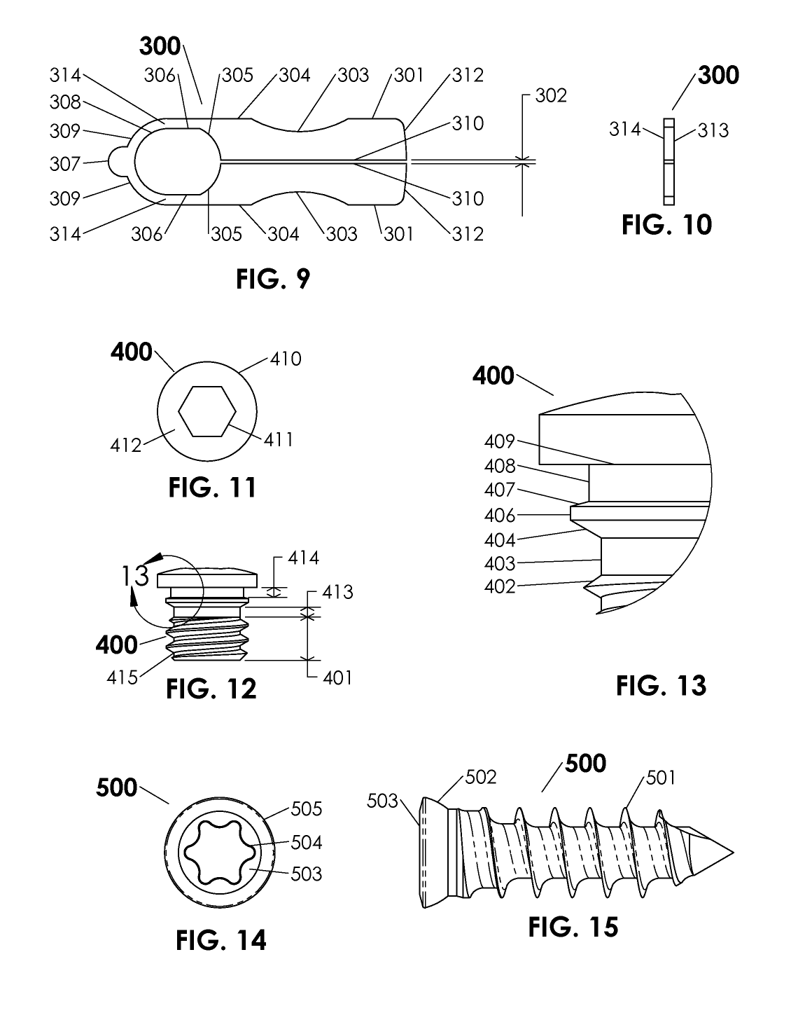

FIG. 1 shows the invention in an anterior cervical plate assembly 100. This is an isometric view and represents how the invention is fully deployed in what is hereafter referred to as the “retention” position.

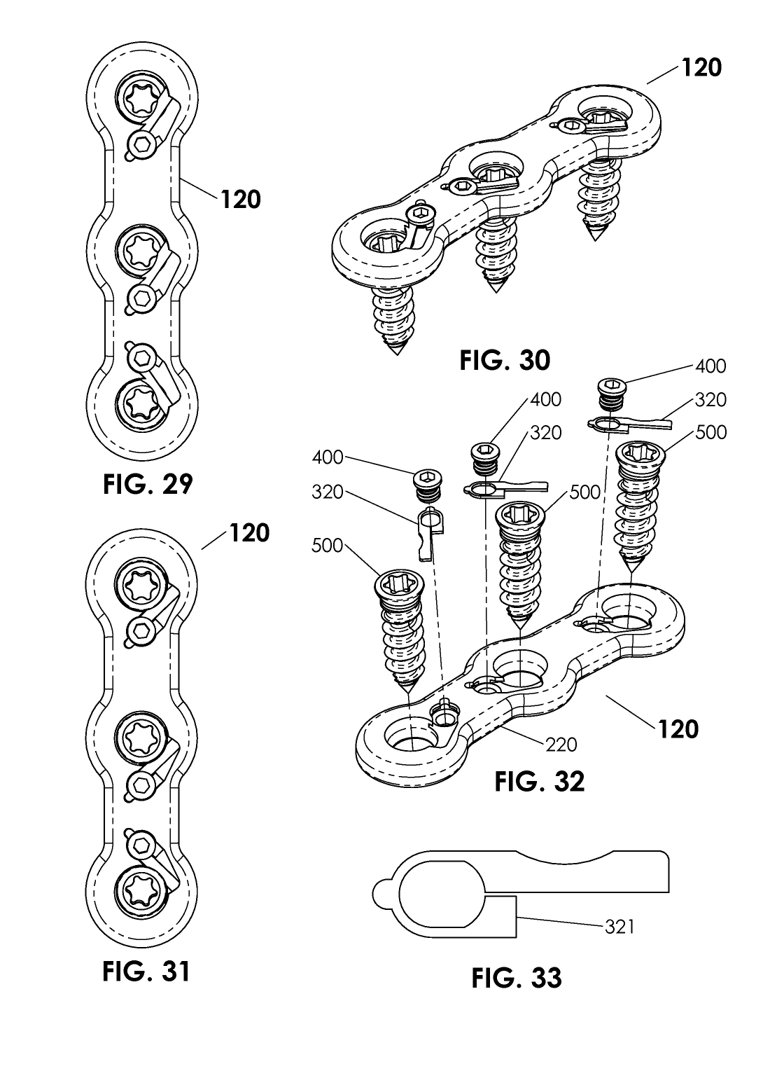

FIG. 2 shows an exploded view of the invention with the following components: the base substrate or “base” (hereafter referenced as an anterior cervical plate 200), a dual-bladed retention blade 300, various retention screws 400 and bone screws 500. In this embodiment of the invention there are three sets of the expanding blade retention system. Each set utilizes one retention blade nest 207 in the base, one dual-bladed retention blade 300, one retention screw 400 and two bone screws 500. It should be understood that two bone screws and a double retention blade are not required for the invention to work. It should be understood that an embodiment using a single bone screw 500 and single bladed retention blade 320, as shown in FIGS. 29-30, will perform the required screw retention.

FIG. 3 is a top view of an anterior cervical plate 200 containing the invention in three separate areas of the plate. The outside contour 201 of the plate is a typical design of a bone plate and has no significant bearing on the invention itself (and similar considerations should be understood for the openings 202 between the screw holes). These surfaces are for visual representations of a typical anterior cervical plate. It should be understood that such shapes and features of the plate need not be present for the invention to function as desired. Also shown in FIG. 3 are three blade nests 207, three female threaded holes 213 to receive the retention screws 400, six bone screw holes 203 and a top surface 210. This view also contains sectional view 5-5 and enlarged view 7 of the anterior cervical plate 200 which are shown in FIGS. 5 and 7 respectively.

FIG. 4 is a side view of the anterior cervical plate 200 and contains partial section 8-8 shown in FIG. 8.

FIG. 5 is an enlarged sectional view along line 5-5 of FIG. 3. The following is a detailed description of one of the six bone screw 500 holes 203. Each hole 203 consists of a conical taper 204, which desirably allows the bone screw 500 to be inserted in and/or positioned at various angles; a spherical diameter 205, which mates with spherical diameter 502 of the bone screw 500 (see FIG. 15) (thereby facilitating the bone screw placement and plate engagement at various angles), and a reduced diameter 206 which desirably prevents the bone screw 500 from passing through the anterior cervical plate 200. Additionally pictured in this view are the underside surface 209 of the anterior cervical plate 200 which desirably rests against the bone, and the two planar surfaces, retention blade nest 207 and upper retention blade surface 208 that capture the dual-bladed retention blade 300 in the anterior cervical plate 200.

FIG. 6 is the bottom view of the anterior cervical plate 200 and shows the bottom surface 209. This bottom surface 209 is placed against the bony surface during surgery.

FIG. 7 is an enlarged top view of area 7 of FIG. 3. This view highlights the top surface 210 (see FIG. 3) of the anterior cervical plate 200 and the contours between the top surface 210 and upper blade retention blade surfaces 208 of one of the three retention blade nests 207. In each retention blade nest 207 there is a female threaded hole 213 that receives the retention screw 400. Also shown in the view is the notch 214 which receives the retention blade tang 307 of the dual-bladed retention blade 300 which desirably secures the central position of the dual retention blade 300. Also shown are two concentric radii 215 which allow for the passage of the retention screw 400 into the retention blade nest 207 and the surface 217 which desirably contains the radial end 312 (see FIG. 9) of the dual retention blade 300.

FIG. 8 is an enlarged sectional view along line 8-8 of FIG. 4. This view is sectioned to show further detail of the retention nest 207. The retention nest 207 has surfaces 218 and, 219 that are desirably offset from surfaces 216 and 217 respectively and surface 220 in order to create a cavity that will retain the dual-bladed retention blade 300 in both the retention and neutral configurations. Surfaces 218, 219 and 220 also create the upper retention blade surface 208 (see FIG. 5). Surfaces 214 and 215 need not necessarily have offset surfaces because they are used to receive the retention screw 400 and retention blade tang 307. Symmetric surfaces 220 are formed to capture the radii 309 of dual-bladed retention blade 300. Symmetric surfaces 218 are formed to capture surfaces 304 of the dual-bladed retention blade 300 when the dual-bladed retention blade 300 is in the retention configuration. Radial surface 207 is formed to capture radial surfaces 312 of the dual-bladed retention blade 300 when the dual-bladed retention blade 300 is either in the retention or neutral configurations.

FIG. 9 is the detailed top view of the dual-bladed retention blade 300. This embodiment of the invention details a dual blade configuration because this embodiment is designed to retain two bone screws 500 simultaneously. It is understood that a single blade embodiment 320, as shown in FIGS. 28, 29, 30 and 31 may operate in the same or a similar manner as the dual blade retention blade 300. It is also understood that any configuration of the retention blade can be manufactured from various types of materials such as, but not limited to, titanium, super-elastic memory metal, polymers or carbon based materials. It is further understood that the manufacturing process used to manufacture the retention blade can be, but not limited to, conventional machining with tool bits, laser cut, water jet or photo etch. The dual blade configuration of the dual-bladed retention blade 300 can include symmetrical surfaces 301, 303, 304, 305, 306, 308, 309 and 310, although non-symmetrical surfaces are contemplated by the present invention as well (see FIG. 33, for example). In this embodiment the surfaces 301 and 304 are collinear and are separated by radius 303. Radius 303 is a clearance for the bone screw 500 when the dual-bladed retention blade 300 is in the neutral configuration. It should be known to those familiar in the art that the retention blade might not need a radius 303 or other indented area in applications where clearance for a component may not be an issue. Where no radius 303 is required, surfaces 301 and 304 may be one continuous surface. It should be also clearly known that the surface defined by surfaces 301, 303 and 304 can have unlimited variation in their features and surfaces as long as the retention of a screw is the intended result. Surfaces 304 and 306 plus surfaces 308 and 309 desirably form flexible arms 314 which flex and/or deform when the dual-bladed retention blade 300 transitions from the neutral to retention configurations and vise-versa. It is understood that the invention need not be limited to four or other number of surfaces to properly operate within the scope of the invention. As described earlier, tab 307 is used to center the dual-bladed retention blade 300 in the retention nest 207 when the dual-bladed retention blade 300 is in either the neutral or retention configurations. There are two surfaces 310 that form a channel or gap 302. It is understood that the invention is not limited to these two parallel surfaces to operate within the scope of the invention as long as there is a separation between the sides. The dual-bladed retention blade 300 is shown in the neutral position or relaxed position. This neutral position is the shape that this embodiment of the dual-bladed retention blade 300 is manufactured to but those familiar in the art can recognize that the retention blades can be manufactured in the retention position and the retention screw could be designed and manipulated in various ways and/or configurations to move the retention blade to the neutral position, if desired.

FIG. 10 is an end view of the dual-bladed retention blade 300 showing top surface 314 and bottom surface 313. It is understood that though the dual-bladed retention blade 300 is shown as two parallel surfaces the invention can have numerous other surfaces or features as mentioned in the description of FIG. 9 and still be within the scope of the invention.

FIG. 11 is the top view of the retention screw 400. In this embodiment, the head diameter 410 is desirably larger than the opening formed by surfaces 305, 306 and 308 of the dual-bladed retention blade 300. The retention screw 400 can be moved, manipulated, driven, twisted and/or rotated by the hex driving feature 411 that is recessed into the head top surface 412. It is understood that the driving feature of the invention is not limited to a hex but may be any driving feature that is commonly known to drive screws.

FIG. 12 is a side view of the retention screw 400 and shows the three areas of the screw 401, 413 and 414. Area 401 is the threaded section that screws into the female threaded holes 213 of the anterior cervical plate 200. Area 413 is the area where the opening formed by surfaces 305, 306 and 308 of the dual-bladed retention blade 300 rests when the dual-bladed retention blade 300 is in the neutral configuration. Area 414 is the area where the opening formed by surfaces 305, 306 and 308 of the dual-bladed retention blade 300 rests when the dual-bladed retention blade 300 is in the retention configuration.

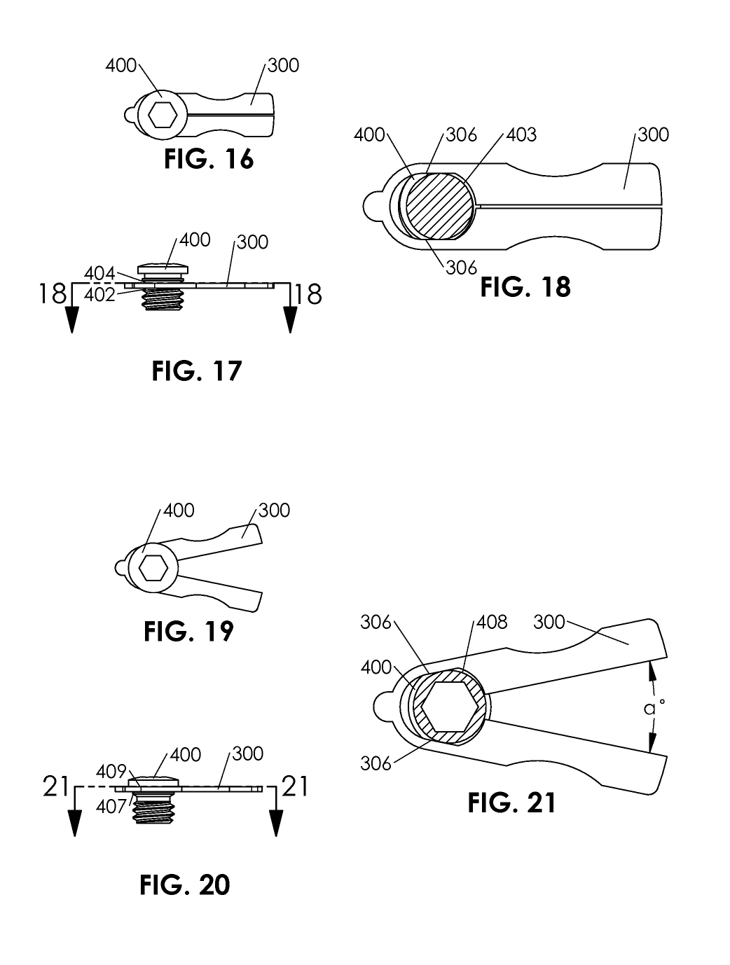

FIG. 13 is an enlarged view of area 13 of the retention screw 400 on FIG. 12. Area 413, shown in FIG. 12, contains diameter 403 and is enclosed by lower neutral conical surface 402 and upper neutral conical surface 404. When the opening formed by surfaces 305, 306 and 308 of the dual-bladed retention blade 300 is encompassing diameter 403 the retention blade is in its unrestrained, or neutral position. The conical surfaces 402 and 404 desirably prevent the dual-bladed retention blade 300 from migrating out of area 413. See detailed descriptions for FIGS. 16, 17 and 18 for further explanation of the neutral relationships between the dual-bladed retention blade 300 and retention screw 400. When the retention screw 400 is advanced by turning or rotating the retention screw 400 with a driver placed in the driving feature 411, the screw can advance into the plate, with the retention blade travelling up the conical surface 404 and onto diameter 406. During this transition from diameter 403 to diameter 406 the channel 302 of the dual-bladed retention blade 300 is spreading open. As the retention screw 400 continues to be driven and/or rotated by the driver in the driving feature 411, the screw can continue advancing into the plate, with the retention blade lower surface 313 dropping down the lower conical retention surface 407 and settling into area 414 and remain in the retention configuration. This transition from diameter 406 to diameter 408 can cause an audible click (in various embodiments) and/or a tactile movement indication can be felt as the retention blade lower surfaces drops onto the diameter 408. Area 414, shown in FIG. 12, contains diameter 408 and is desirably enclosed by lower retention conical surface 407 and shoulder 409. When the opening formed by surfaces 305, 306 and 308 of the dual-bladed retention blade 300 is encompassing diameter 408 the retention blade is a “restrained” or retention position. The conical surfaces 407 and shoulder 409 can desirably prevent the dual-bladed retention blade 300 from migrating out of area 414, if desired. See detailed descriptions for FIGS. 19, 20 and 21 for further explanation of the retention relationships between the dual-bladed retention blade 300 and retention screw 400.

FIG. 14 is the top view of one exemplary embodiment of a bone screw 500. Head diameter 505 is desirably larger than the reduced diameter 206 (or “necked” portion) of the anterior cervical plate 200, which desirably prevents the screw from advancing through the anterior cervical plate 200. The bone screw 500 can be driven by the hexalobe or star driving feature 504 that is recessed into the head top surface 503. It is understood that the driving feature of the invention is not limited to a hexalobe but may be any driving feature that is commonly known to drive screws.

FIG. 15 is the side view of the bone screw 500 and is one of substantially generic design. Only the significant features of the bone screw that directly interact with the invention will be detailed here. The thread diameter 501 can be smaller than the reduced diameter 206 of the anterior cervical plate 200 (if desired) to allow unimpeded passage of the bone screw 500 through the anterior cervical plate 200. Spherical diameter 502 mates with the spherical diameter 205 of the anterior cervical plate 200, thereby allowing for angular placement of the bone screw 500. In various embodiments, the top surface 503 will desirably contact the dual-bladed retention blade 300 when it is in the retention configuration, thereby retaining the bone screw 500 and prevent it from backing out of the anterior cervical plate 200. See detailed descriptions for FIGS. 25, 26 and 27 for further explanation of the retaining feature and the relationships between the dual-bladed retention blade 300 and bone screw 500.

FIG. 16 is the top view of a partial assembly of the dual-bladed retention blade 300 and retention screw 400, in the neutral configuration, in order to demonstrate the working relationship between the two components.

FIG. 17 is the side view of a partial assembly of the dual-bladed retention blade 300 and retention screw 400, in the neutral configuration, in order to demonstrate the working relationship between the two components. In this embodiment, the dual-bladed retention blade 300 is nestled in the area 413 (see FIG. 12) of the retention screw 400 between conical surfaces 402 and 404.

FIG. 18 is an enlarged sectional view of the dual-bladed retention blade and retention screw defined by the line 18-18 from FIG. 17. The diameter 403 of the retention screw 400 is shown between the surfaces 306 of the dual-bladed retention blade 300. The dual-bladed retention blade 300 is shown in the neutral position.

FIG. 19 is the top view of a partial assembly of the dual-bladed retention blade 300 and retention screw 400, in the retention configuration, in order to demonstrate the working relationship between the two components.

FIG. 20 is the side view of a partial assembly of the dual-bladed retention blade 300 and retention screw 400, in the retention configuration, in order to demonstrate the working relationship between the two components. The retention blade 300 is contacting the enlarged diameter 408 (see FIG. 12) of the retention screw 400 between the shoulder 407 and conical surface 409.

FIG. 21 is an enlarged sectional view defined by the line 21-21 from FIG. 20. The diameter 408 of the retention screw 400 is shown between the surfaces 306 of the dual-bladed retention blade 300. The dual-bladed retention blade 300 is shown in the retention position. Because diameter 408 is larger than the smaller diameter 403 portion of the retention screw 400, the retention blade is forced open to angle α.

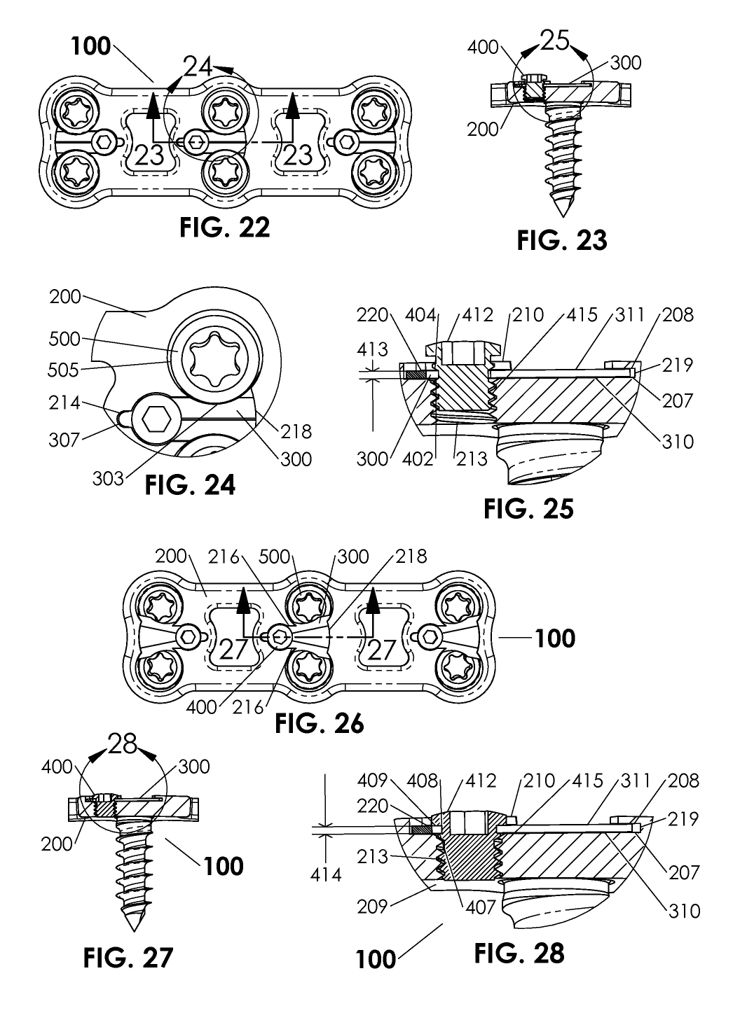

FIG. 22 is the top view of the anterior cervical plate assembly 100 in the neutral position and contains sectional view line 23-23 and view area 24.

FIG. 23 is a cross-sectional view of the anterior cervical plate assembly 100 defined by the line 23-23 from FIG. 22, which sections the anterior cervical plate 200, dual-bladed retention blade 300 and retention screw 400.

FIG. 24 is an enlarged partial view of area 24 of the of the anterior cervical plate assembly 100 from FIG. 22. The dual-bladed retention blade 300 is shown in the neutral configuration where the radius 303 is positioned so the head diameter 505 of the bone screw 500 can travel past the dual-bladed retention blade 300 without hindrance. Also shown is the tang 307 of the dual-bladed retention blade 300 placed into the notch 214 of the anterior cervical plate 200. Though not completely shown the end surface 312 (see FIG. 9) of the dual-bladed retention blade 300 is captured inside the area defined by radius 218, surface 207, surface 208 and radius 219 of the anterior cervical plate (see FIG. 25).

FIG. 25 is an enlarged view of area 25 of the anterior cervical plate assembly 100 from FIG. 23, which sections the anterior cervical plate 200, dual-bladed retention blade 300 and retention screw 400 in the neutral position. The top surface 412 of the retention screw 400 is shown above the top surface 210 of the anterior cervical plate. The surfaces 310 and 311 of the dual-bladed retention blade 300 are shown between the conical surfaces 402 and 404 and in area 413 of the retention screw 400. The surfaces 310 and 311 of the dual-bladed retention blade 300 are shown captured between surfaces 208 and 207 of the anterior cervical plate 200 at radii 219 and 220. One feature of the invention is that these areas at the radii 219 and 220 retain the dual-bladed retention blade 300 which in turn retains the retention screw because it is constrained by the conical surfaces 402 and 404 and the mating threads 213 and 415. This feature allows the dual-bladed retention blade 300 and retention screw 400 to be preassembled before the surgery, thereby reducing surgery time.

FIG. 26 is the top view of the anterior cervical plate assembly 100 in the retention position and contains sectional view line 27-27. Though not completely shown the end surface 312 (see FIG. 9) of the dual-bladed retention blade 300 is inside the area defined by radius 218, surface 207, surface 208 and radius 219 of the anterior cervical plate and under the surfaces (see FIG. 9).

FIG. 27 is a sectional view of the anterior cervical plate assembly 100 defined by the line 27-27 from FIG. 26 and sections the anterior cervical plate 200, dual-bladed retention blade 300 and retention screw 400.

FIG. 28 is an enlarged view of area 28 of the anterior cervical plate assembly 100 from FIG. 27 and sections the anterior cervical plate 200, dual-bladed retention blade 300 and retention screw 400 in the retention position. The top surface 412 of the retention screw 400 is now shown just above the top surface 210 of the anterior cervical plate, although the top surface 412 of the retention screw 400 could similarly be at (i.e., flush with) or below the top surface 210 of the cervical plate 200 (see FIGS. 1 and 30, for example) without hindering the intended function of the retention screw 400. The surfaces 310 and 311 of the dual-bladed retention blade 300 are shown between the conical surface 407 and shoulder 409 and in area 414 of the retention screw 400. The surfaces 310 and 311 of the dual-bladed retention blade 300 are shown captured between surfaces 208 and 207 of the anterior cervical plate 200 at radii 219 and 220 and also under surfaces 216. Another feature of the invention is that these areas at the radii 219 and 220 retain the dual-bladed retention blade 300 which in turn retains the retention screw 400 because it is constrained by the conical surface 407, planar surface 409 and the mating threads 213 and 415. This feature holds the assembly in the retention position and does not absolutely require the retention screw 400 to be torqued tightly to prevent the retention screw from backing out since it is retained in position by the dual-bladed retention blade 300.

FIG. 32 is an exploded isometric view of another embodiment of an anterior cervical plate 220 containing a single-bladed embodiment 120 of the invention in the neutral position. For the single-bladed configuration the retention screw 400 and bone screw 500 features can remain the same, if desired, however the anterior cervical plate 220 shown now has a smaller nest than the retention blade nest 207 of the embodiment 100 because the retention feature has only a one-sided retention blade 320 while the prior embodiment of the invention depicted a dual-bladed symmetric design 300.

FIG. 33 is a top view of another alternative embodiment of a retention blade, specifically a single-bladed retention blade. Instead of the symmetric bladed design of the dual-bladed retention blade 300, the single blade retention blade 320 includes a shorter leg portion 321 that serves a similar functional purpose as the longer leg of the dual-bladed retention blade 300, but including a smaller profile.

Those who are skilled in the art understand that the profile of the single blade retention blade 320 can vary in the same manner as the dual bladed retention blade 300, as previously described, as long as the retention of a screw is the intended result.

All references, including any publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader, and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A screw retention system comprising:
a base for attachment to a bony surface of a patient, the base including an upper surface and a lower surface and at least one through-hole for accommodating a bone screw, the base further including a recessed surface proximate to the through-hole for accommodating a retention blade and a blade retention screw therein;
the bone screw including a threaded portion that is sized and configured to pass through the through-hole and into the bony surface, the bone screw having a head portion that is larger than a minimal diameter of the through-hole; and
the blade retention screw including a first reduced diameter portion and a second increased diameter portion, wherein when the first reduced diameter portion is in contact with the retention blade the retention blade assumes a first position allowing advancement of the bone screw into the through-hole, but when the second increased diameter portion is in contact with the retention blade the retention blade assumes a second position that hinders movement of the bone screw out of the through-hole.

2. The screw retention system of claim 1, wherein the retention blade comprises a flexible material.

3. The screw retention system of claim 1, wherein the retention blade comprises an elastic material.

4. The screw retention system of claim 1, wherein the retention blade includes a first elongated arm and a second elongated arm that are connected together near a proximal end of the blade, with an opening formed between the first and second arms that is sized and configured to accommodate the blade retention screw.

5. The screw retention system of claim 1, wherein the base includes a plurality of through-holes for accommodating a plurality of bone screws.

6. The screw retention system of claim 4, wherein the first elongated arm is positioned proximate to a first through-hole in the base for accommodating a first bone screw, and the second elongated arm is positioned proximate to a second through-hole in the base for accommodating a second bone screw, the first and second elongated arms being operable to retain the first and second bone screws in the base by rotation of the blade retention screw.

7. The screw retention system of claim 1, wherein rotation of the blade retention screw moves the retention blade from the first position to the second position.

8. The screw retention system of claim 5, wherein a single retention blade includes a plurality of elongated arms operable for retaining the plurality of bone screws.

9. The screw retention system of claim 1, wherein an upper surface of the blade retention screw extends above the upper surface of the base.

10. The screw retention system of claim 1, wherein an upper surface of the blade retention screw is recessed below the upper surface of the base.

11. The screw retention system of claim 1, wherein the retention blade includes a single elongated arm operable between the first and second positions.

12. The screw retention system of claim 5, wherein the base comprises a surgical plate for promoting bone fusion between two bony structures.

13. An apparatus for promoting bone fusion between two adjacent bony structures, the apparatus comprising:
a base having a top surface and a bottom surface opposite the top surface, the base having a plurality of through-holes extending from the top surface to the bottom surface, the plurality of through-holes sized and configured to accommodate a plurality of bone screws for attachment to the two adjacent bony structures;
a bone screw retention mechanism disposed within a recess formed into the top surface of the base, the bone screw retention mechanism including an elongated member at least partially secured into the recess by a blade retention screw, the elongated member operable between a first position and second position by rotation of the blade retention screw, wherein in the first position the elongated member extends over at least a portion of a bone screw positioned within a through-hole and, wherein in the second position the elongated member does not extend over at least a portion of the bone screw.

14. The apparatus of claim 13, wherein the elongated member comprises a flexible material.

15. The apparatus of claim 13, wherein the elongated member comprises an elastic material.

16. The apparatus of claim 13, wherein the recess formed into the top surface of the base further includes a peripheral cavity sized and configured to retain at least a portion of a peripheral edge of the elongated member within the peripheral cavity when the elongated member is moved between the first and second positions.

17. The apparatus of claim 13, wherein the elongated member includes a first elongated arm and a second elongated arm that are connected together near a proximal end of the elongated member, with an opening formed between the first and second arms that is sized and configured to accommodate the blade retention screw.

18. The apparatus of claim 17, wherein the first elongated arm is positioned proximate to a first through-hole of the plurality of through-holes, and the second elongated arm is positioned proximate to a second through-hole of the plurality of through-holes, the first and second elongated arms being operable to retain a first bone screw and a second bone screw in the first and second through-holes by rotation of the blade retention screw.

19. The apparatus of claim 13, wherein an upper surface of the elongated member is recessed below the top surface of the base.

20. The apparatus of claim 13, wherein rotation of the blade retention screw positions a lower surface of the elongated member into contact with an upper surface of the bone screw.
